(19) United States
(12) Patent Application Publication
(10) Pub. No.: US 2008/0287542 A1
(43) Pub. Date: Nov. 20, 2008

(54) Novel Isovaleramide Forms, Compositions Thereof, and Related Methods of Use

(76) Inventors: Mark Oliveira, Bedford, MA (US); Matthew Peterson, Hopkinton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/912,759

(22) PCT Filed: Apr. 27, 2006
(86) PCT No.: PCT/US2006/016151

§ 371 Date: Aug. 1, 2008

Related U.S. Application Data
(60) Provisional application No. 60/675,606, filed on Apr. 28, 2005.

(51) Int. Cl. A61K 31/195; A61P 25/24; A61P 25/18; A61P 25/00

(52) U.S. Cl. 514/563

(57) ABSTRACT

The invention provides novel isovaleramide forms. These forms include isovaleramide co-crystals, and solvates, hydrates, co-crystals, and polymorphs thereof. The invention also provides novel compositions comprising these novel forms and one or more suitable carriers. The invention also provides related methods of treatment or prevention. Compositions and methods of the invention have a number of uses, including the treatment or prevention of epilepsy and anxiety.

### FIELD OF THE INVENTION

The invention provides novel isovaleramide forms. These forms include isovaleramide co-crystals, and solvates, hydrates, co-crystals, and polymorphs thereof.

The invention also provides novel pharmaceutical compositions comprising these novel forms and related methods of treatment or prevention.

Compositions and methods of the invention are useful in the treatment or prevention of a number of conditions including sleep disorders.

### BACKGROUND OF THE INVENTION

Isovaleramide is a valproic acid analogue that has the chemical name 3-methylbutyramide. The molecular formula for isovaleramide is C₅H₁₁NO, which corresponds to a molecular weight of 101.15. Isovaleramide has the structure of Formula I:

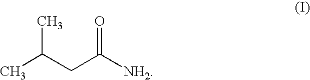

Isovaleramide is useful for the treatment or prevention of a number of conditions, including for example epilepsy, migraine, Parkinson's disease, bipolar disorder, depression, schizophrenia, CNS disorders, and anxiety.

Notwithstanding the current availability of any isovaleramide forms (e.g., polymorphs, salts, solvates, and hydrates), the need continues to exist for isovaleramide forms that evidence improved properties, such as for example aqueous solubility and stability, and thereby enable the manufacture and use of a broad range of safe and effective isovaleramide pharmaceutical dosage forms.

### SUMMARY OF THE INVENTION

The invention provides novel forms of isovaleramide. These forms include novel co-crystals of isovaleramide free base, and solvates, hydrates, co-crystals, and polymorphs thereof. In certain embodiments, novel isovaleramide forms of the invention are readily formulated, exhibit improved dissolution, improved stability, modulated dose response, decreased hygroscopicity, and/or exhibit improved aqueous solubility when compared to known isovaleramide forms.

The invention also provides novel pharmaceutical compositions comprising these novel forms of isovaleramide and related methods of treatment or prevention.

Compositions and methods of the invention are useful in the treatment or prevention of epilepsy, migraine, Parkinson's disease, bipolar disorder, depression, schizophrenia, CNS disorders, and anxiety, for example.

In one illustrative embodiment, the invention provides a novel isovaleramide form formed by the reaction of isovaleramide and a carboxylic acid. For example, the invention provides a novel isovaleramide form formed by the combination of isovaleramide with citric acid, gentisic acid, glutaric acid, maleic acid, or mandelic acid.

In certain embodiments, the isovaleramide forms can cause less or no discoloration of solid pharmaceutical dosage forms than known isovaleramide forms; and/or can be more water-soluble than known isovaleramide forms.

These and other embodiments of the invention are described further in the detailed description of the invention.

### BRIEF DESCRIPTION OF THE FIGURES

FIG. 1A illustrates powder X-ray diffraction (PXRD) measurements of an isovaleramide:citric acid co-crystal.

FIG. 1B illustrates a Fourier transform infrared (IR) measurement of an isovaleramide:citric acid co-crystal.

FIG. 1C illustrates Raman spectroscopic measurements of an isovaleramide:citric acid co-crystal.

FIG. 2A illustrates the differential scanning calorimetry (DSC) measurements of an isovaleramide:gentisic acid co-crystal taken from approximately room temperature to 200° C. at 10° C./minute.

FIG. 2B illustrates a thermogravimetric analysis (TGA) of an isovaleramide:gentisic acid co-crystal taken from approximately room temperature to approximately 400° C. at 10° C./minute.

FIG. 2C illustrates an IR spectroscopy measurement of an isovaleramide:gentisic acid co-crystal.

FIG. 2D illustrates PXRD measurements of an isovaleramide:gentisic acid co-crystal.

FIG. 2E illustrates Raman spectroscopic measurements of an isovaleramide:gentisic acid co-crystal.

FIG. 2F illustrates PXRD measurements of an isovaleramide:gentisic acid co-crystal.

FIG. 2G illustrates the DSC measurements of an isovaleramide:gentisic acid co-crystal taken from approximately room temperature to 200° C. at 10° C./minute.

FIG. 2H illustrates an IR spectroscopy measurement of an isovaleramide:gentisic acid co-crystal.

FIG. 2I illustrates Raman spectroscopic measurements of an isovaleramide:gentisic acid co-crystal.

FIG. 3A illustrates the DSC measurements of an isovaleramide:glutaric acid co-crystal taken from approximately room temperature to 200° C. at 10° C./minute.

FIG. 3B illustrates an IR spectroscopy measurement of an isovaleramide:glutaric acid co-crystal.

FIG. 3C illustrates PXRD measurements of an isovaleramide:glutaric acid co-crystal.

FIG. 3D illustrates a Raman spectroscopic measurement of an isovaleramide:glutaric acid co-crystal.

FIG. 4A illustrates the DSC measurements of an isovaleramide:maleic acid co-crystal taken from approximately room temperature to 200° C. at 10° C./minute.

FIG. 4B illustrates an IR spectroscopy measurement of an isovaleramide:maleic acid co-crystal.

FIG. 4C illustrates PXRD measurements of an isovaleramide:maleic acid co-crystal.

FIG. 4D illustrates a Raman spectroscopic measurement of an isovaleramide:maleic acid co-crystal.

FIG. 5A illustrates the DSC measurements of an isovaleramide:mandelic acid co-crystal taken from approximately room temperature to 200° C. at 10° C./minute.

FIG. 5B illustrates a TGA of an isovaleramide:mandelic acid co-crystal taken from approximately room temperature to approximately 400° C. at 10° C./minute.

FIG. 5C illustrates the IR spectroscopy measurement of an isovaleramide:mandelic acid co-crystal.

FIG. 5D illustrates PXRD measurements of an isovaleramide:mandelic acid co-crystal.

FIG. 5E illustrates a Raman spectroscopic measurement of an isovaleramide:mandelic acid co-crystal.

FIG. 5F illustrates the DSC measurements of an isovaleramide:mandelic acid co-crystal taken from approximately room temperature to 200° C. at 10° C./minute.

FIG. 5G illustrates PXRD measurements of an isovaleramide:mandelic acid co-crystal.

### DETAILED DESCRIPTION OF THE INVENTION

“Carboxylic acids” include, but are not limited to, formic, acetic, propionic, butyric, isobutyric, valeric, isovaleric, pivalic, caproic, caprylic, capric, lauric, myristic, palmitic, stearic, acrylic, crotonic, benzoic, cinnamic, mandelic, gentisic, and salicylic acids. Throughout the specification and the claims, the term “carboxylic acid” can be used to specifically include both carboxylic acids and dicarboxylic acids. Similarly, the term “carboxylic acid” can also be used to include any molecule which comprises at least one —COOH functional group.

“Dicarboxylic acid” includes, in certain embodiments, a compound of formula (II):

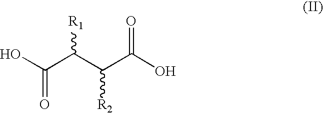

wherein R₁ and R₂ are each independently H, OH, Cl, Br, I, substituted or unsubstituted C₁₋₆ alkyl, substituted or unsubstituted aryl or R₁ and R₂ taken together represent a double bond as well as stereochemically pure D or L salts of a compound of formula (II). The term “dicarboxylic acid” is herein defined as a subclass of the broader class known as “carboxylic acid.”

Examples of the dicarboxylic acid of formula (II) include but are not limited to succinic acid, maleic acid, tartaric acid, malic acid, or fumaric acid. Dicarboxylic acids of formula (II) that can be used to make compounds of the invention include, e.g., succinic acid, tartaric acid, malic acid, and fumaric acid. Dicarboxylic acids in addition to those of Formula II, such as malonic acid and adipic acid, can also be used. Dicarboxylic acids can be in the form of a substantially pure (R)-(+)-enantiomer; a substantially pure (R)-(−)-enantiomer; a substantially pure (S)-(+)-enantiomer; or a substantially pure (S)-(−)-enantiomer.

“Co-crystal” as used herein means a crystalline material comprised of two or more unique solids at room temperature, each containing distinctive physical characteristics, such as structure, melting point, and heats of fusion, with the exception that, if specifically stated, the API (active pharmaceutical ingredient) may be a liquid at room temperature. The co-crystals of the present invention comprise a co-crystal former H-bonded to isovaleramide or a derivative thereof. The co-crystal former may be H-bonded directly to isovaleramide or may be H-bonded to an additional molecule which is bound to isovaleramide. The additional molecule may be H-bonded to isovaleramide or bound ionically or covalently to isovaleramide. The additional molecule could also be a different API. Solvates of isovaleramide compounds that do not further comprise a co-crystal former are not co-crystals according to the present invention. The co-crystals may however, include one or more solvate molecules in the crystalline lattice. That is, solvates of co-crystals, or a co-crystal further comprising a solvent or compound that is a liquid at room temperature, are included in the present invention, but crystalline material comprised of only isovaleramide and one or more liquids (at room temperature) are not included. Other modes of molecular recognition may also be present including, pi-stacking, guest-host complexation, and van der Waals interactions. Of the interactions listed above, hydrogen-bonding is the dominant interaction in the formation of the co-crystal, (and a required interaction according to the present invention) whereby a non-covalent bond is formed between a hydrogen bond donor of one of the moieties and a hydrogen bond acceptor of the other. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads. An alternative embodiment provides for a co-crystal wherein the co-crystal former is a second API. In another embodiment, the co-crystal former is not an API. For purposes of the present invention, the chemical and physical properties of isovaleramide in the form of a co-crystal may be compared to a reference compound that is isovaleramide in a different form. The reference compound may be specified as a free form, or more specifically, an anhydrate or hydrate of a free form, or a solvate of a free form. For example, the reference compound for isovaleramide in free form co-crystallized with a co-crystal former can be isovaleramide in free form. The reference compound may also be specified as crystalline or amorphous. The reference compound may also be specified as the most stable polymorph of the specified form of the reference compound.

The terms “pharmaceutical composition” and “medicament” are used herein interchangeably.

In a first embodiment, the present invention provides co-crystals of isovaleramide, and methods of making and using the same. In another embodiment, the present invention provides isovaleramide co-crystals further comprising at least one organic acid, and methods of making and using the same. In another embodiment, the present invention provides isovaleramide co-crystals further comprising at least one carboxylic acid, and methods of making and using the same.

In one embodiment, the present invention is directed to a novel co-crystal comprising isovaleramide and citric acid.

In certain embodiments, the co-crystal comprising isovaleramide and citric acid is a co-crystal wherein the isovaleramide and the citric acid are present in a ratio of about 10:1 to about 1:10; in a ratio of about 5:1 to about 1:5; about 2:1 to about 1:2; in a ratio of about 1.1:1 to about 1:1.1; or in a ratio of about 1:1.

The isovaleramide:citric acid co-crystal can be characterized according to a number of physical features. One such feature is the powder X-ray diffraction pattern of the isovaleramide:citric acid co-crystal. In certain embodiments, the isovaleramide:citric acid co-crystal is characterized as having one, two, three, or four or more powder X-ray diffraction peaks including, but not limited to, 5.0, 15.3, 15.7, and 20.2 degrees 2-theta. In another embodiment, the isovaleramide:citric acid co-crystal is characterized as having four or more powder X-ray diffraction peaks including, but not limited to, 5.0, 15.3, 15.7, and 20.2 degrees 2-theta.

In yet another embodiment, the isovaleramide:citric acid co-crystal is characterized as having a PXRD peak at approximately 5.0 degrees 2-theta.

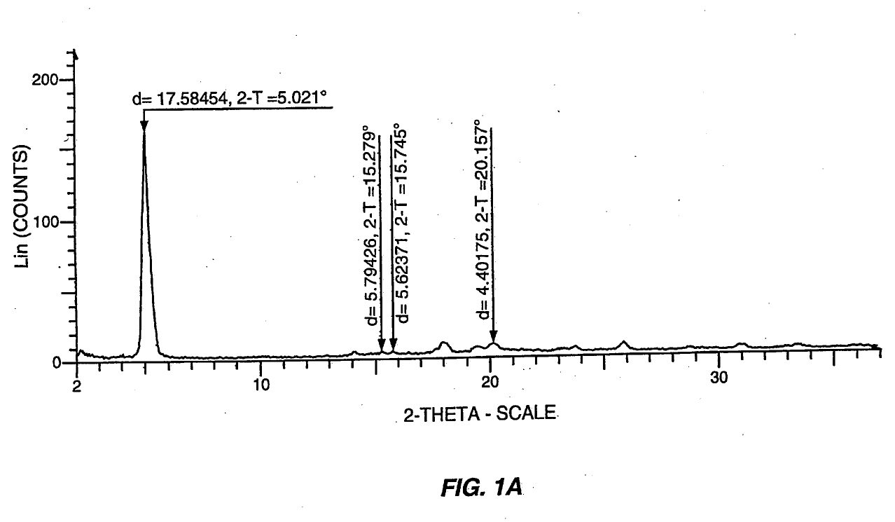

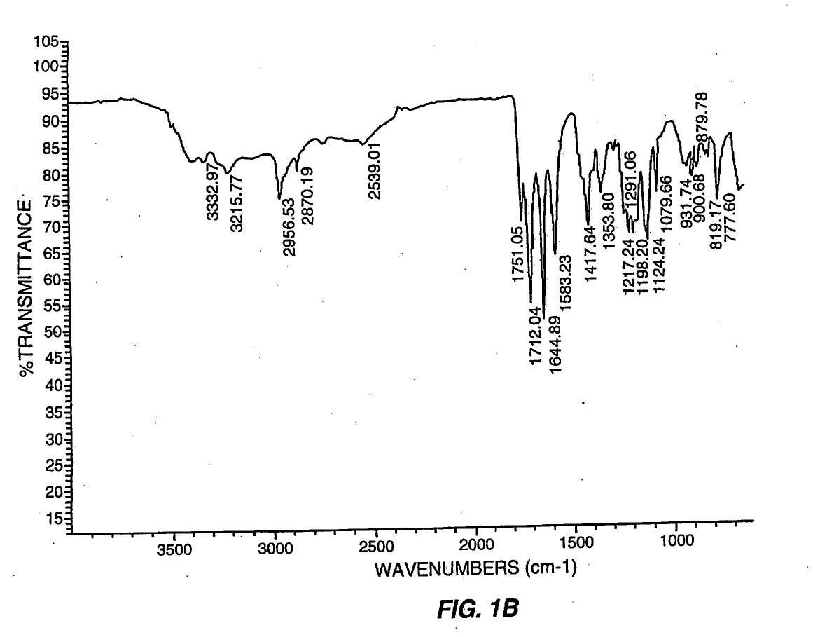

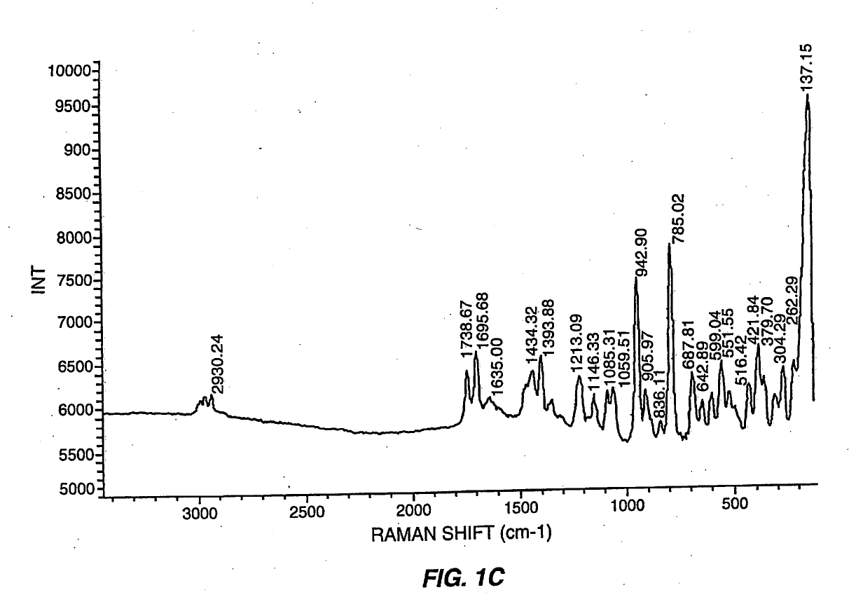

In a further embodiment, the isovaleramide:citric acid co-crystal has a powder X-ray diffraction pattern that is substantially the same as the pattern shown in FIG. 1A.

A further physical characteristic of the isovaleramide:citric acid co-crystal of the present invention includes its Raman spectrum. For example, certain embodiments of the isovaleramide:citric acid co-crystal of the present invention are characterized as having a Raman spectrum comprising peaks at about 2930, 1739, 1696, 1635, 1434, 1394, 1213, 1146, 1085, 1060, 943, 906, 836, 785, 688, 643, 599, 552, 516, 422, 380, 304, 262, and 137 cm⁻¹.

In a further embodiment, the isovaleramide:citric acid co-crystal has a Raman spectroscopic pattern that is substantially the same as the pattern shown in FIG. 1C.

A further physical characteristic of the isovaleramide:citric acid co-crystal of the present invention includes its Fourier-transform infrared (FTIR or IR) spectrum. For example, certain embodiments of the isovaleramide:citric acid co-crystal of the present invention are characterized as having an IR spectrum comprising peaks at about 3333, 3216, 2957, 2870, 1751, 1712, 1645, 1583, 1418, 1354, 1217, 1198, 1124, 1080, 932, 901, 880, 819, and 778 cm⁻¹. In another embodiment, the isovaleramide:citric acid co-crystal is characterized as having a IR spectrum that is substantially the same as the pattern shown in FIG. 1B.

The isovaleramide:citric acid co-crystal in certain embodiments is about 95.00%, 96.00%, 97.00%, 98.00%, 99.00%, 99.90%, or 99.99% free of impurities including solvate molecules.

The citric acid used in the co-crystal may be anhydrous citric acid.

In another embodiment, the present invention is directed to a novel co-crystal comprising isovaleramide and gentisic acid.

In certain embodiments, the co-crystal comprising isovaleramide and gentisic acid is a co-crystal wherein the isovaleramide and the gentisic acid are present in a ratio of about 10:1 to about 1:10; in a ratio of about 5:1 to about 1:5; about 2:1 to about 1:2; in a ratio of about 1.1:1 to about 1:1.1; or in a ratio of about 1:1.

The isovaleramide:gentisic acid co-crystal can be characterized according to a number of physical features. One such feature is the powder X-ray diffraction pattern of the isovaleramide:gentisic acid co-crystal. In certain embodiments, the isovaleramide:gentisic acid co-crystal is characterized as having at least one, two, three, or four or more powder X-ray diffraction peaks including, but not limited to, 7.5, 12.9, 15.1, 16.1, 17.5, 18.3, 19.3, 23.2, 24.4, and 25.8 degrees 2-theta.

In another embodiment, the isovaleramide:gentisic acid co-crystal is characterized as having five or more powder X-ray diffraction peaks including, but not limited to, 7.5, 12.9, 15.1, 16.1, 17.5, 18.3, 19.3, 23.2, 24.4, and 25.8 degrees 2-theta. In another embodiment, the isovaleramide:gentisic acid co-crystal is characterized as having a powder X-ray diffractogram comprising peaks at about 7.5, 12.9, 15.1, 16.1, 17.5, 18.3, 19.3, 21.0, 23.2, 24.4, 25.8, 27.5, 28.2, 28.8, 30.6, 31.5, 35.3, 36.0, and 38.8 degrees 2-theta.

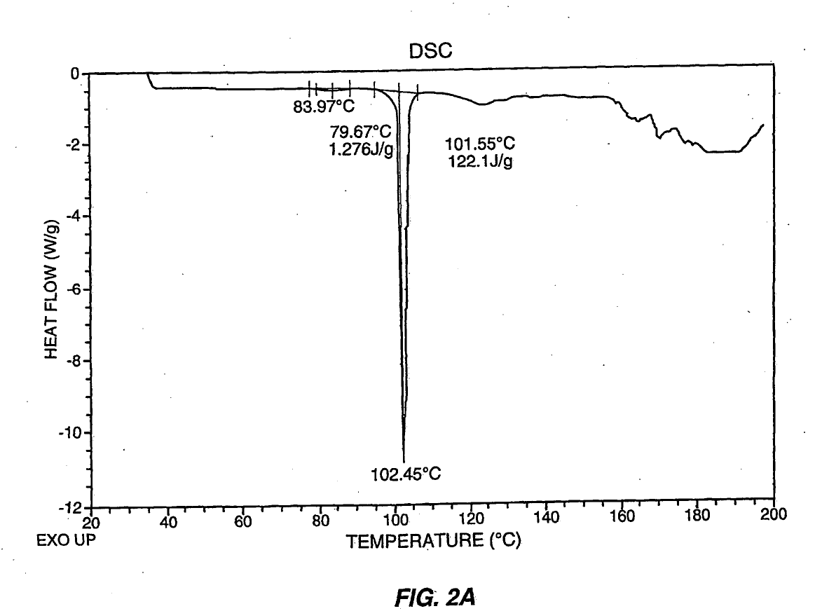

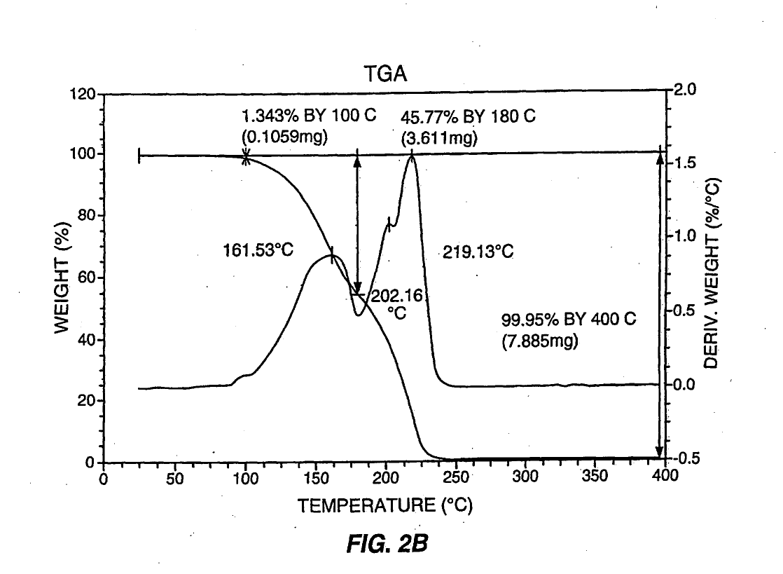

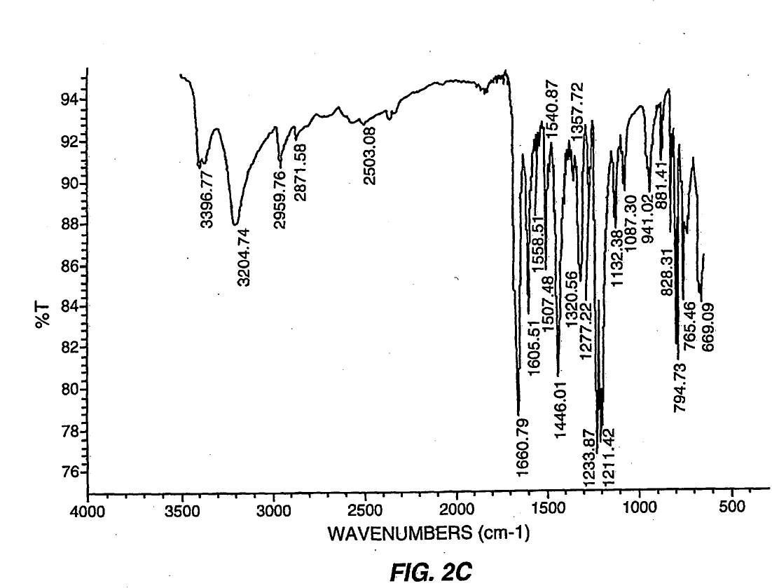

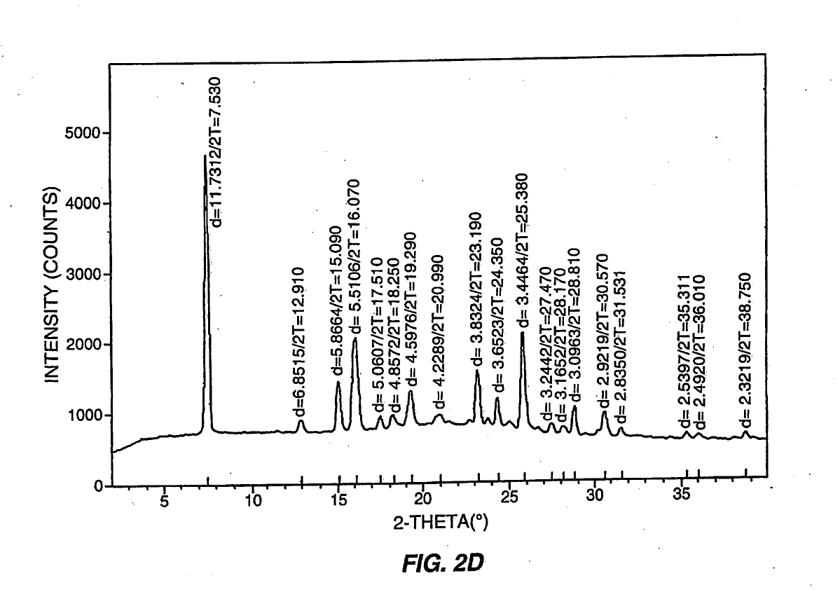

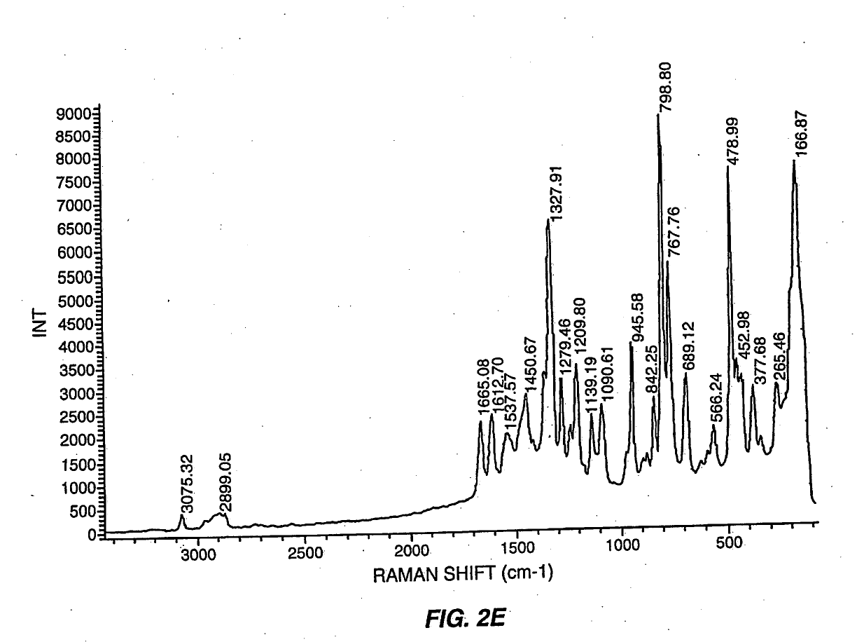

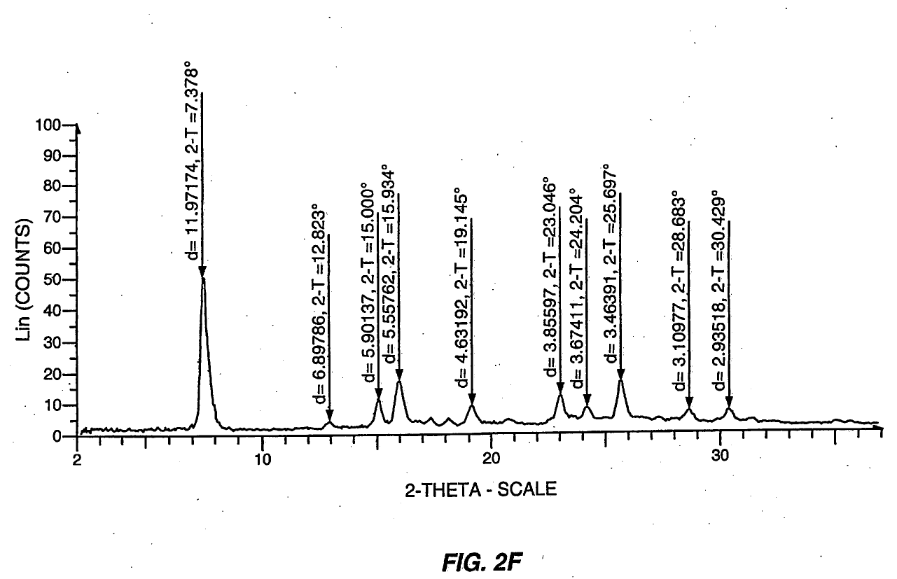

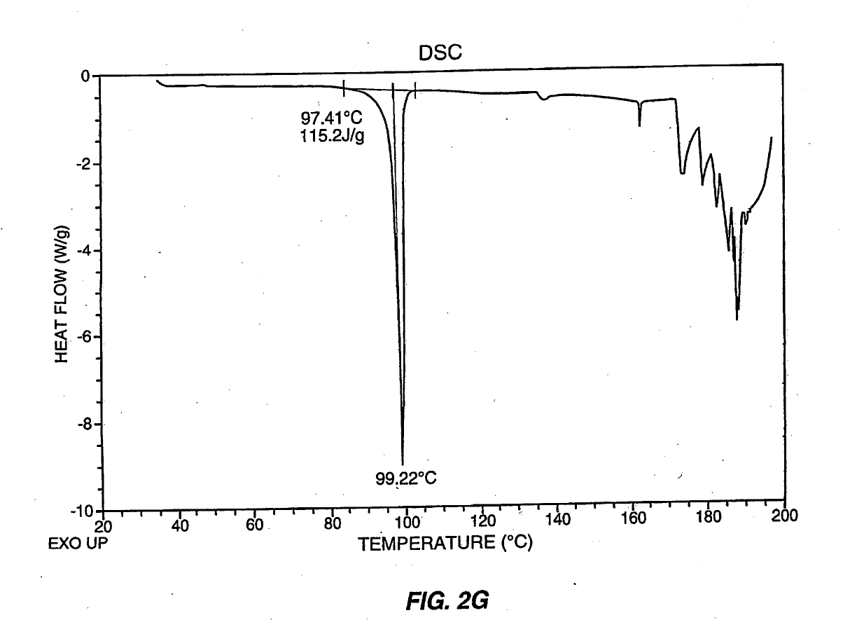

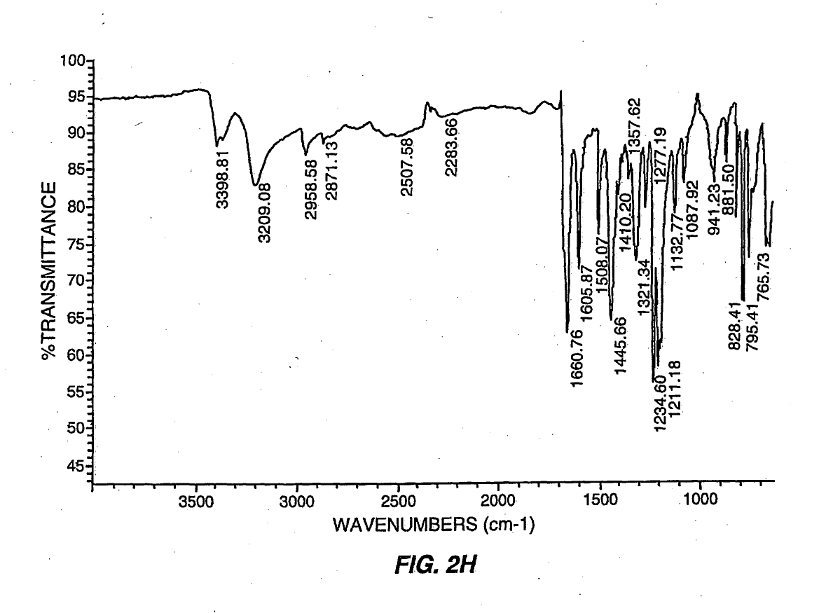

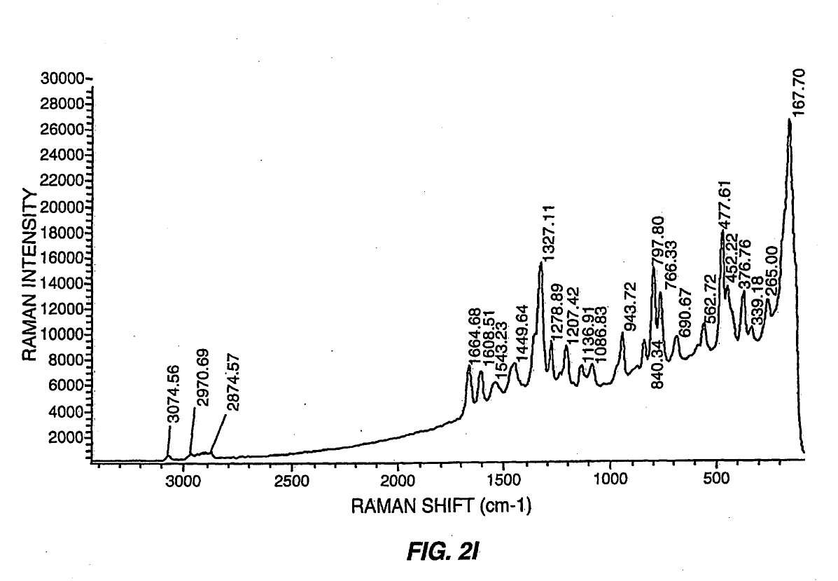

In a further embodiment, the isovaleramide:gentisic acid co-crystal has a powder X-ray diffraction pattern that is substantially the same as the pattern shown in FIG. 2D.

In other embodiments, the isovaleramide:gentisic acid co-crystal is characterized as having one, two, three, or four or more powder X-ray diffraction peaks including, but not limited to, 7.4, 12.8, 15.0, 15.9, 19.1, 23.0, 24.2, and 25.7 degrees 2-theta. In another embodiment, the isovaleramide:gentisic acid co-crystal is characterized as having five or more powder X-ray diffraction peaks including, but not limited to, 7.4, 12.8, 15.0, 15.9, 19.1, 23.0, 24.2, and 25.7 degrees 2-theta. In another embodiment, the isovaleramide:gentisic acid co-crystal is characterized as having a powder X-ray diffractogram comprising peaks at about 7.4, 12.8, 15.0, 15.9, 19.2, 23.1, 24.2, 25.7, 28.7, and 30.4 degrees 2-theta.

In a further embodiment, the isovaleramide:gentisic acid co-crystal has a powder X-ray diffraction pattern that is substantially the same as the pattern shown in FIG. 2F.

A further physical characteristic of the isovaleramide:gentisic acid co-crystal of the present invention includes its Raman spectrum. For example, certain embodiments of the isovaleramide:gentisic acid co-crystal of the present invention are characterized as having a Raman spectrum comprising peaks at about 3075, 2899, 1665, 1612, 1538, 1451, 1328, 1279, 1210, 1139, 1091, 946, 842, 799, 768, 689, 566, 479, 453, 378, 265, and 169 cm⁻¹.

In a further embodiment, the isovaleramide:gentisic acid co-crystal has a Raman spectrum that is substantially the same as the pattern shown in FIG. 2E.

Other embodiments of the isovaleramide:gentisic acid co-crystal of the present invention are characterized as having a Raman spectrum comprising peaks at about 3075, 2971, 2875, 1665, 1609, 1543, 1450, 1327, 1279, 1207, 1137, 1087, 944, 840, 798, 766, 691, 563, 478, 452, 377, 339, 265, and 168 cm⁻¹.

In a further embodiment, the isovaleramide:gentisic acid co-crystal has a Raman spectrum that is substantially the same as the pattern shown in FIG. 2I.

A further physical characteristic of the isovaleramide:gentisic acid co-crystal of the present invention includes its IR spectrum. For example, certain embodiments of the isovaleramide:gentisic acid co-crystal of the present invention are characterized as having an IR spectrum comprising peaks at about 3397, 3205, 2960, 2872, 2503, 1661, 1606, 1559, 1541, 1507, 1446, 1358, 1321, 1277, 1234, 1211, 1132, 1087, 941, 881, 828, 795, 765, and 669 cm⁻¹. In another embodiment, the isovaleramide:gentisic acid co-crystal is characterized as having a IR spectrum that is substantially the same as the pattern shown in FIG. 2C.

Other embodiments of the isovaleramide:gentisic acid co-crystal of the present invention are characterized as having an IR spectrum comprising peaks at about 3399, 3209, 2959, 2871, 2508, 2284, 1661, 1606, 1508, 1446, 1410, 1358, 1321, 1277, 1235, 1211, 1133, 1088, 941, 882, 828, 795, and 766 cm⁻¹. In another embodiment, the isovaleramide:gentisic acid co-crystal is characterized as having a IR spectrum that is substantially the same as the pattern shown in FIG. 2H.

In yet a further embodiment, the isovaleramide:gentisic acid co-crystal is characterized as having an endothermic transition at about 102 degrees C. In yet a further embodiment, the isovaleramide:gentisic acid co-crystal is characterized as having an endothermic transition of about 99 degrees C. In one embodiment, an isovaleramide:gentisic acid co-crystal exhibits a DSC scan substantially as shown in FIG. 2A or 2G.

Certain embodiments of an isovaleramide:gentisic acid co-crystal exhibit a weight loss of about 45% over a temperature range from about 30 degrees C. to about 180 degrees C., as determined by TGA. In an alternative aspect, the isovaleramide:gentisic acid co-crystal is characterized as exhibiting a weight loss, as determined by TGA, substantially similar to that shown in FIG. 2B.

The isovaleramide:gentisic acid co-crystal in certain embodiments is about 95.00%, 96.00%, 97.00%, 98.00%, 99.00%, 99.90%, or 99.99% free of impurities including solvate molecules.

In another embodiment, the present invention is directed to a novel co-crystal comprising isovaleramide and glutaric acid.

In certain embodiments, the co-crystal comprising isovaleramide and glutaric acid is a co-crystal wherein the isovaleramide and the glutaric acid are present in a ratio of about 10:1 to about 1:10; in a ratio of about 5:1 to about 1:5; in a ratio of about 2:1 to about 1:2; in a ratio of about 1.1:1 to about 1:1.1; or in a ratio of about 1:1.

The isovaleramide:glutaric acid co-crystal can be characterized according to a number of physical features. One such feature is the powder X-ray diffraction pattern of the isovaleramide:glutaric acid co-crystal. In certain embodiments, the isovaleramide:glutaric acid co-crystal is characterized as having one, two, three, or four or more powder X-ray diffraction peaks including, but not limited to, 8.3, 9.1, 19.7, 21.6, 22.8, and 23.5 degrees 2-theta. In another embodiment, the isovaleramide:glutaric acid co-crystal is characterized as having five or more powder X-ray diffraction peaks including, but not limited to, 8.3, 9.1, 19.7, 21.6, 22.8, and 23.5 degrees 2-theta.

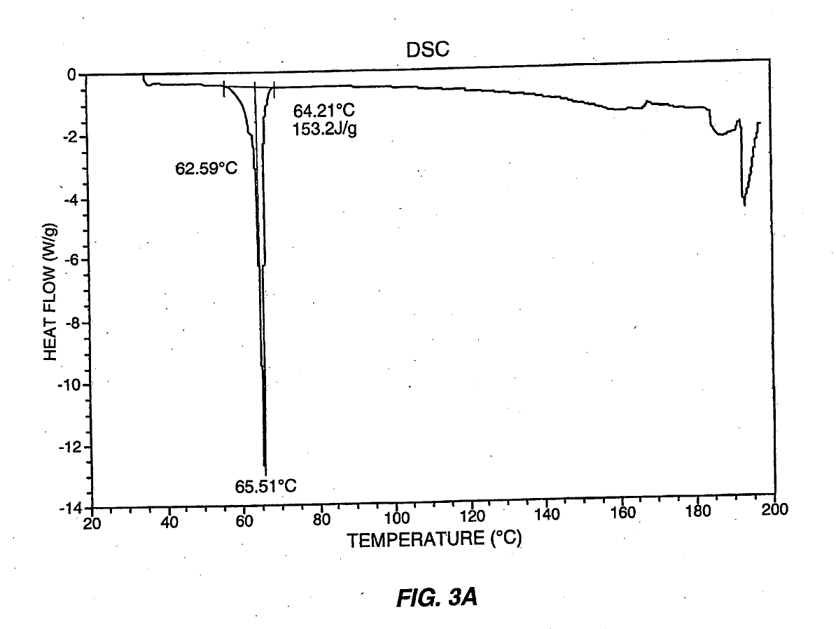

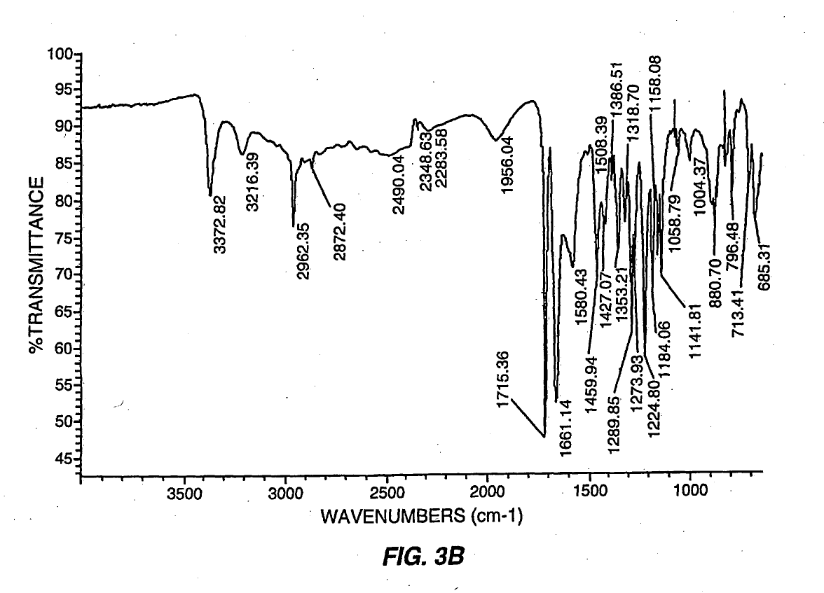

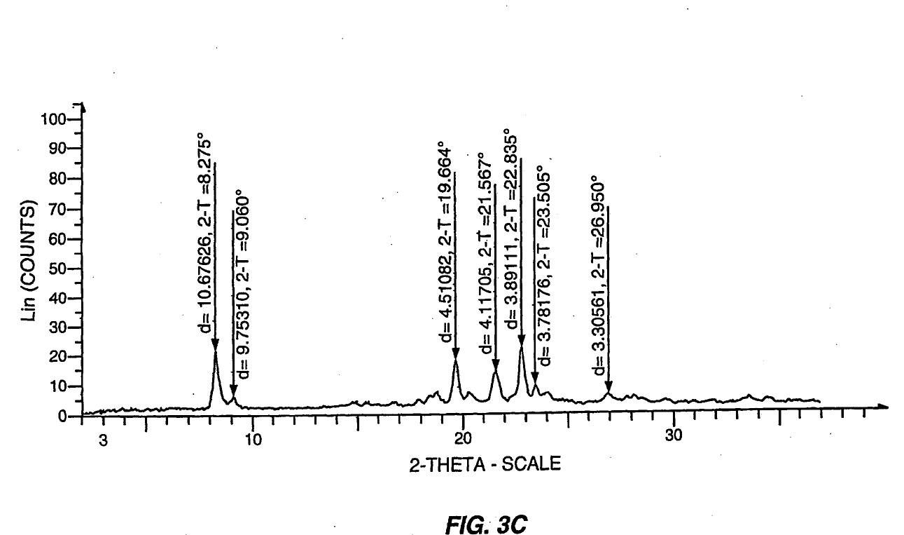

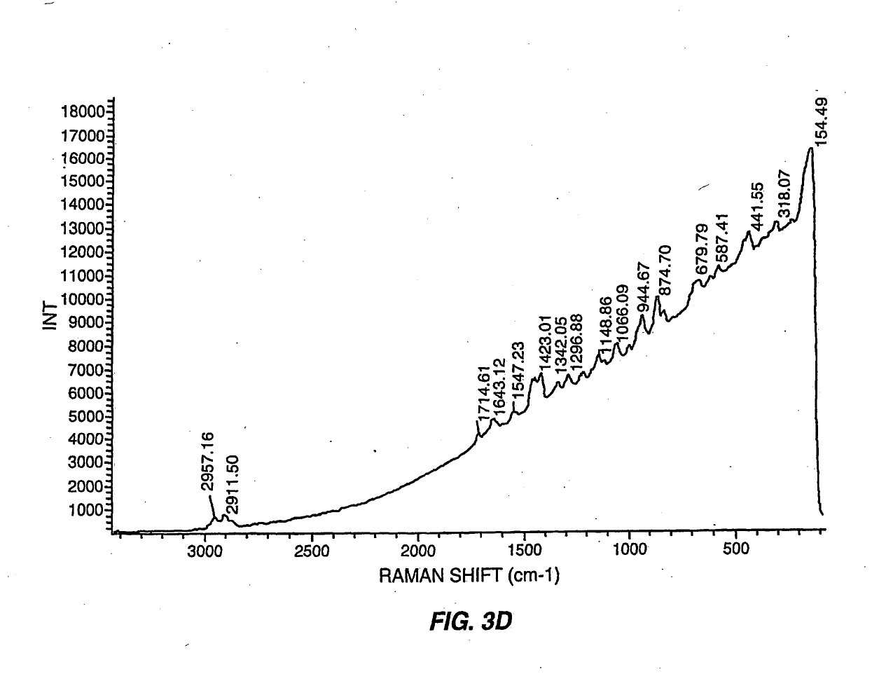

In a further embodiment, the isovaleramide:glutaric acid co-crystal has a powder X-ray diffraction pattern that is substantially the same as the pattern shown in FIG. 3C.

A further physical characteristic of the isovaleramide:glutaric acid co-crystal of the present invention includes its Raman spectrum. For example, certain embodiments of the isovaleramide:glutaric acid co-crystal of the present invention are characterized as having a Raman spectrum comprising peaks at about 2957, 2912, 1715, 1643, 1547, 1423, 1342, 1297, 1149, 1066, 945, 875, 680, 587, 442, 318, and 154 cm⁻¹.

In a further embodiment, the isovaleramide:glutaric acid co-crystal has a Raman spectrum that is substantially the same as the pattern shown in FIG. 3D.

A further physical characteristic of the isovaleramide:glutaric acid co-crystal of the present invention includes its IR spectrum. For example, certain embodiments of the isovaleramide:glutaric acid co-crystal of the present invention are characterized as having an IR spectrum comprising peaks at about 3373, 3216, 2962, 2872, 2490, 2349, 2284, 1956, 1715, 1661, 1580, 1508, 1460, 1427, 1387, 1353, 1319, 1290, 1274, 1225, 1184, 1158, 1142, 1059, 1004, 881, 796, 713, and 685 cm⁻¹. In another embodiment, the isovaleramide:glutaric acid co-crystal is characterized as having an IR spectrum that is substantially the same as the pattern shown in FIG. 3B.

In yet a further embodiment, the isovaleramide:glutaric acid co-crystal is characterized as having an endothermic transition at about 66° C. For example, an isovaleramide:glutaric acid co-crystal exhibits a DSC scan substantially as shown in FIG. 3A.

The isovaleramide:glutaric acid co-crystal in certain embodiments is about 95.00%, 96.00%, 97.00%, 98.00%, 99.00%, 99.90%, or 99.99% free of impurities including solvate molecules.

In another embodiment, the present invention is directed to a novel co-crystal comprising isovaleramide and maleic acid.

In certain embodiments, the co-crystal comprising isovaleramide and maleic acid is a co-crystal wherein the isovaleramide and the maleic acid are present in a ratio of about 10:1 to about 1:10; in a ratio of about 5:1 to about 1:5; about 2:1 to about 1:2; in a ratio of about 1.1:1 to about 1:1.1; or in a ratio of about 1:1.

The isovaleramide:maleic acid co-crystal can be characterized according to a number of physical features. One such feature is the powder X-ray diffraction pattern of the isovaleramide:maleic acid co-crystal. In certain embodiments, the isovaleramide:maleic acid co-crystal is characterized as having one, two, three, or four or more powder X-ray diffraction peaks including, but not limited to, 8.6, 18.5, 20.2, 20.4, 21.5, 23.0, and 26.2 degrees 2-theta. In another embodiment, the isovaleramide:maleic acid co-crystal is characterized as having five or more powder X-ray diffraction peaks including, but not limited to, 8.6, 18.5, 20.2, 20.4, 21.5, 23.0, and 26.2 degrees 2-theta. In another embodiment, the isovaleramide:maleic acid co-crystal is characterized as having a powder X-ray diffractogram comprising peaks at about 8.6, 17.3, 17.8, 18.5, 20.2, 20.4, 21.5, 23.0, 26.2, and 26.9 degrees 2-theta.

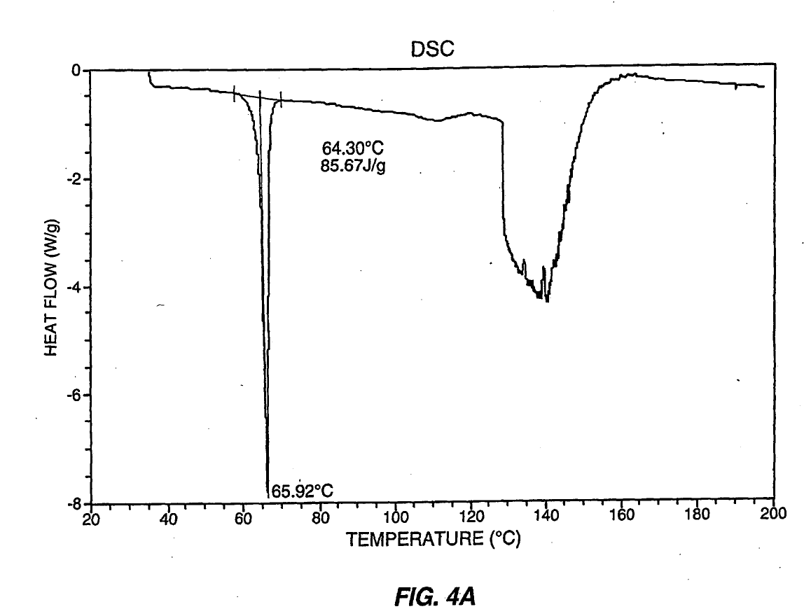

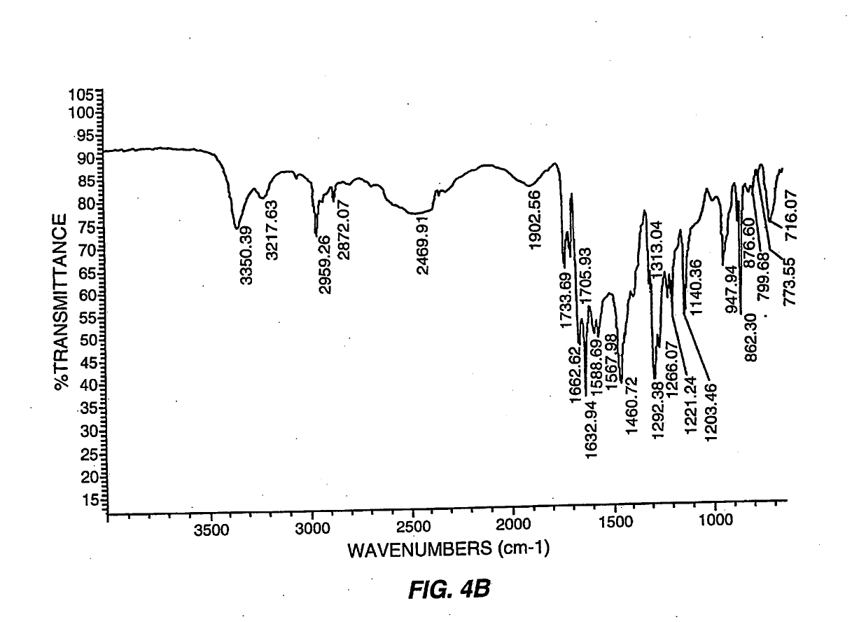

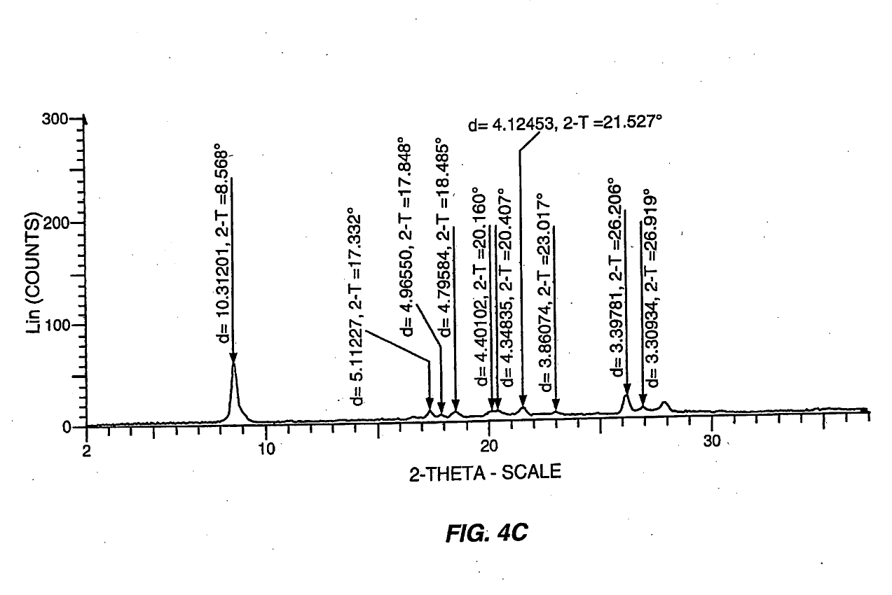

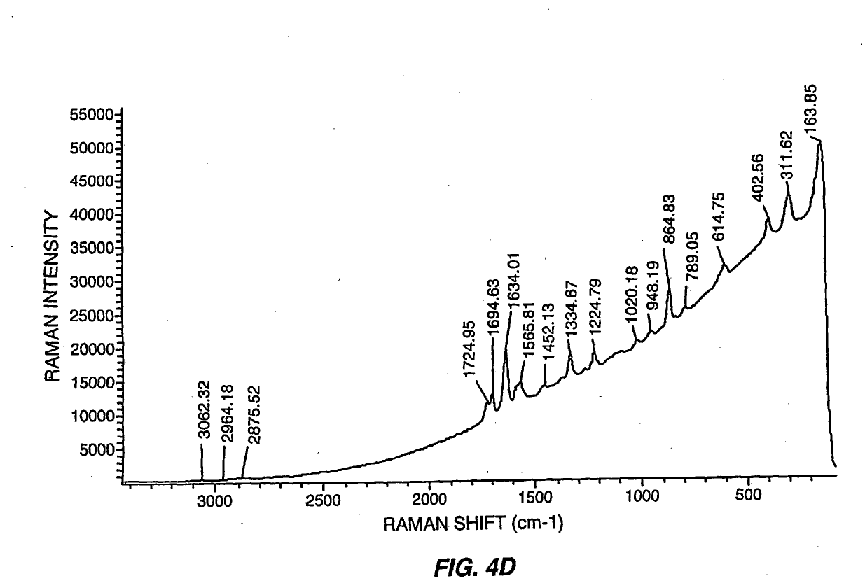

In a further embodiment, the isovaleramide:maleic acid co-crystal has a powder X-ray diffraction pattern that is substantially the same as the pattern shown in FIG. 4C.

A further physical characteristic of the isovaleramide:maleic acid co-crystal of the present invention includes its Raman spectrum. For example, certain embodiments of the isovaleramide:maleic acid co-crystal of the present invention are characterized as having a Raman spectrum comprising peaks at about 3062, 2964, 2876, 1725, 1695, 1634, 1566, 1452, 1335, 1225, 1020, 948, 865, 789, 615, 403, 312, and 164 cm⁻¹.

In a further embodiment, the isovaleramide:maleic acid co-crystal has a Raman spectrum that is substantially the same as the pattern shown in FIG. 4D.

A further physical characteristic of the isovaleramide:maleic acid co-crystal of the present invention includes its IR spectrum. For example, certain embodiments of the isovaleramide:maleic acid co-crystal of the present invention are characterized as having an IR spectrum comprising peaks at about 3350, 3218, 2959, 2872, 2470, 1903, 1734, 1706, 1663, 1633, 1589, 1568, 1461, 1313, 1292, 1266, 1221, 1203, 1140, 948, 877, 862, 800, 774, and 716 cm⁻¹. In another embodiment, the isovaleramide:maleic acid co-crystal is characterized as having an IR spectrum that is substantially the same as the pattern shown in FIG. 4B.

In yet a further embodiment, the isovaleramide:maleic acid co-crystal is characterized as having an endothermic transition at about 66° C. For example, an isovaleramide:maleic acid co-crystal exhibits a DSC scan substantially as shown in FIG. 4A.

The isovaleramide:maleic acid co-crystal in certain embodiments is about 95.00%, 96.00%, 97.00%, 98.00%, 99.00%, 99.90%, or 99.99% free of impurities including solvate molecules.

In yet another embodiment, the present invention is directed to a novel co-crystal form comprising isovaleramide and mandelic acid.

In certain embodiments, the co-crystal comprising isovaleramide and mandelic acid is a co-crystal wherein the isovaleramide and the mandelic acid are present in a ratio of about 10:1 to about 1:10; in a ratio of about 5:1 to about 1:5; about 2:1 to about 1:2, in a ratio of about 1.1:1 to about 1:1.1; or in a ratio of about 1:1.

The isovaleramide:mandelic acid co-crystal can be characterized according to a number of physical features. One such feature is the powder X-ray diffraction pattern of the isovaleramide:mandelic acid co-crystal. In certain embodiments, the isovaleramide:mandelic acid co-crystal is characterized as having one, two, three, or four or more powder X-ray diffraction peaks including, but not limited to, 6.6, 10.1, 17.2, 17.8, 20.6, 21.2, 24.8, and 26.7 degrees 2-theta. In another embodiment, the isovaleramide:mandelic acid co-crystal is characterized as having five or more powder X-ray diffraction peaks including, but not limited to, 6.6, 10.1, 17.2, 17.8, 20.6, 21.2, 24.8, and 26.7 degrees 2-theta. In another embodiment, the isovaleramide:mandelic acid co-crystal is characterized as having a powder X-ray diffractogram comprising peaks at about 6.6, 10.1, 17.2, 17.8, 18.5, 20.0, 20.6, 21.2, 24.8, 25.4, 25.7, 26.7, 30.0, and 34.7 degrees 2-theta.

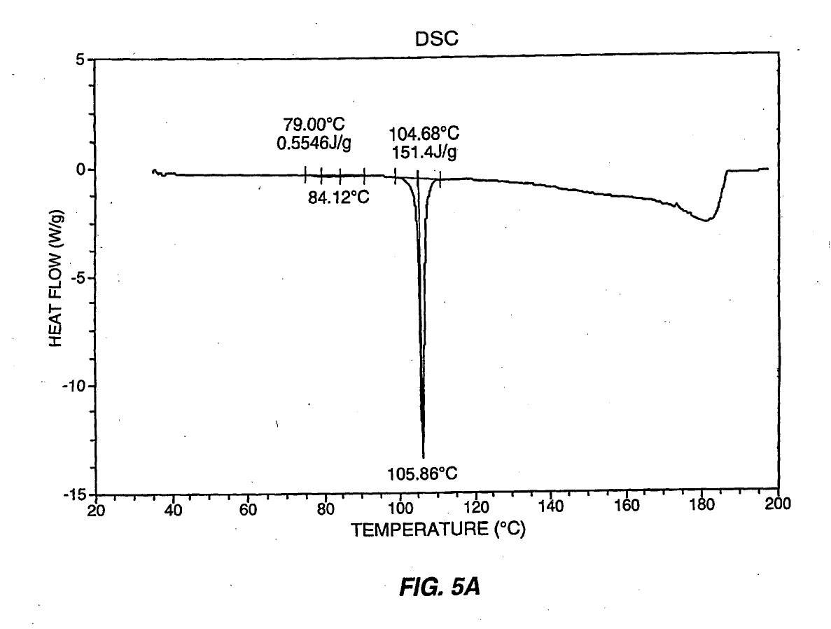

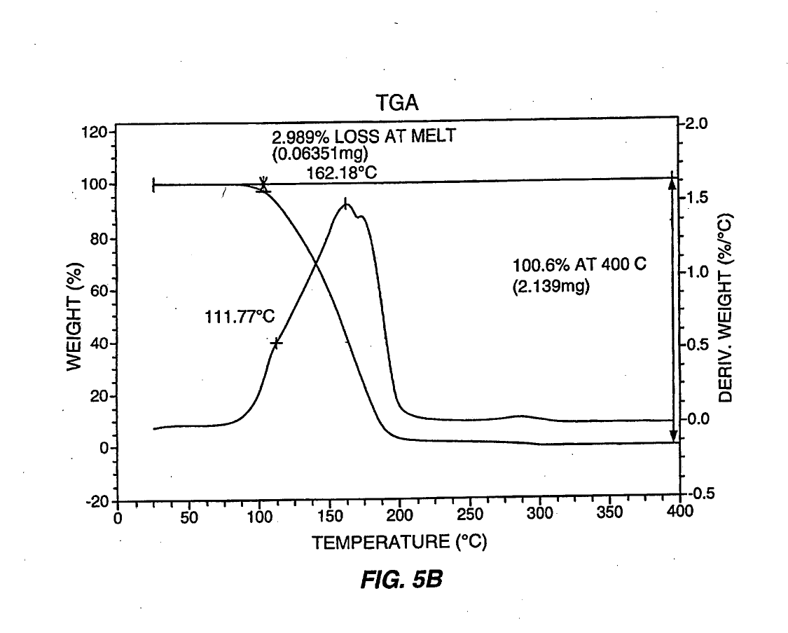

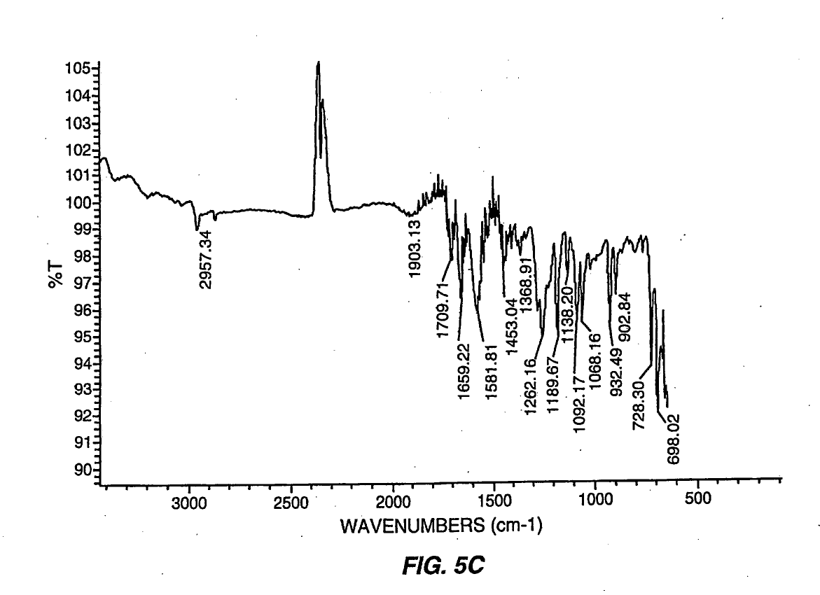

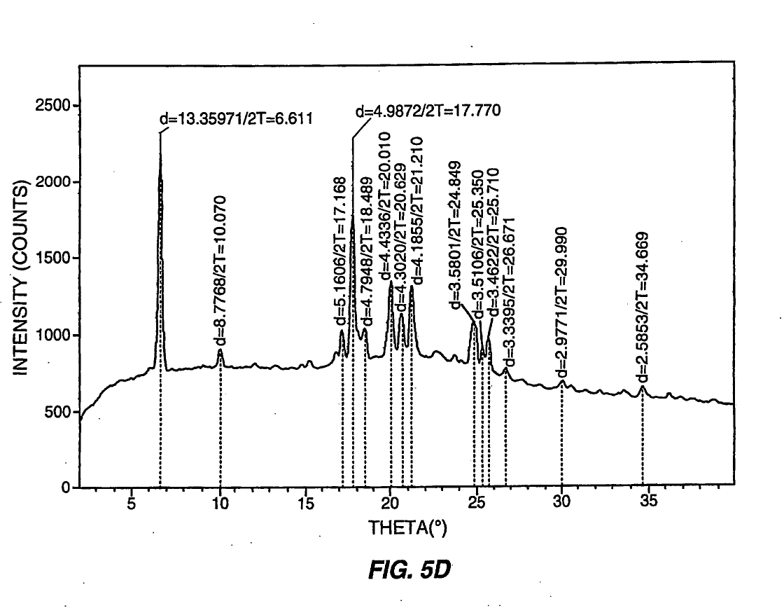

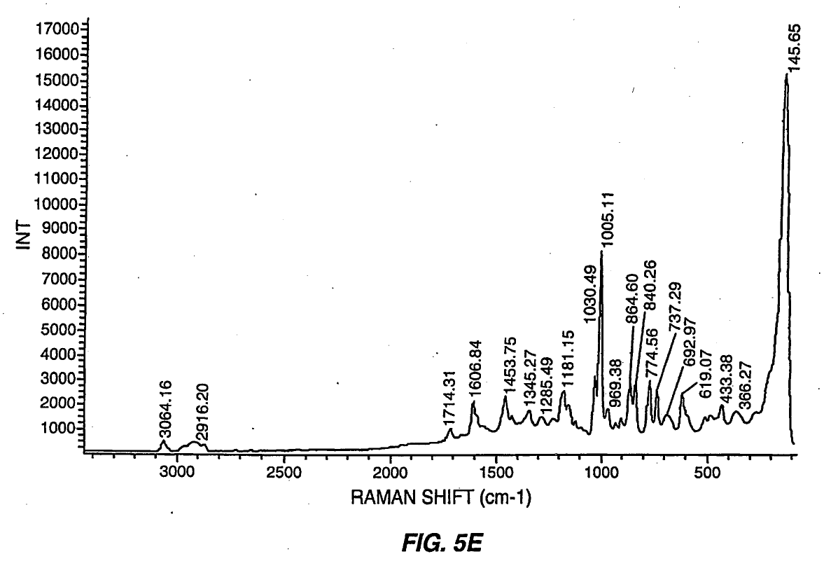

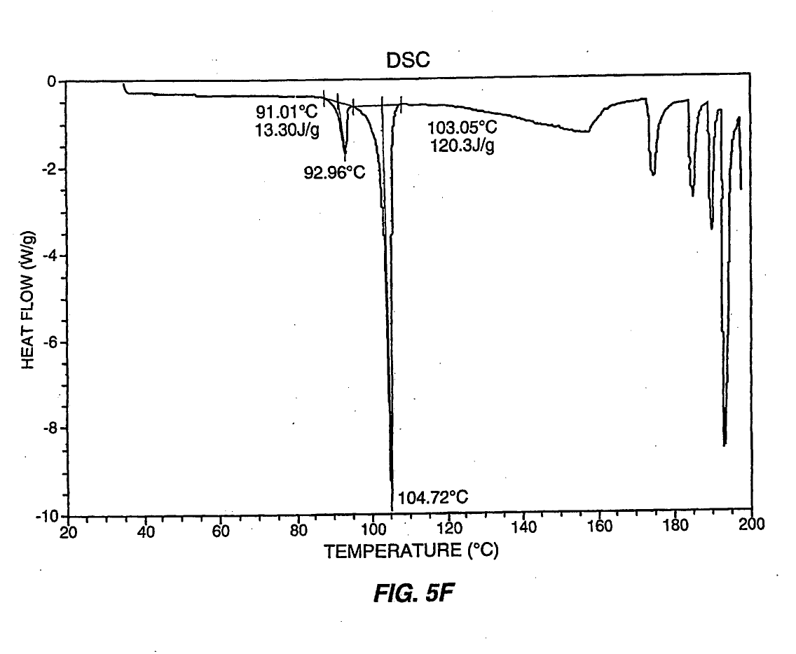

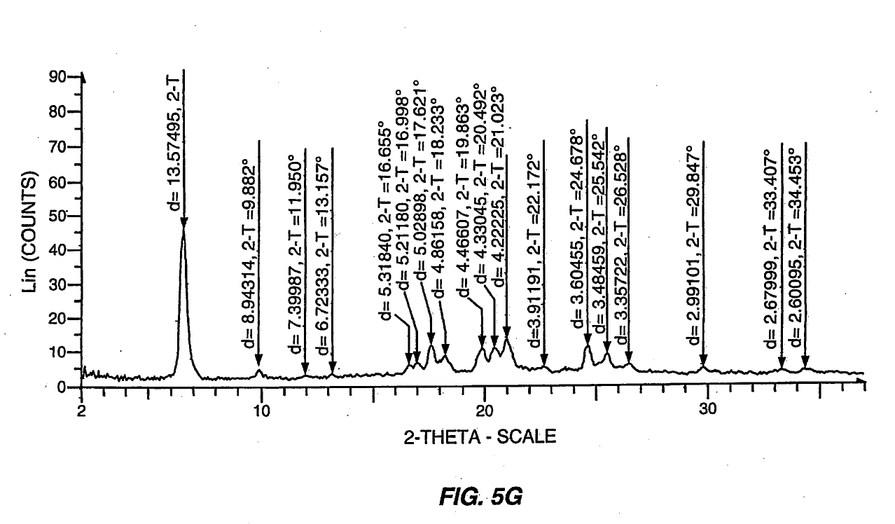

In a further embodiment, the isovaleramide:mandelic acid co-crystal has a powder X-ray diffraction pattern that is substantially the same as the pattern shown in FIG. 5D.

In certain embodiments, the isovaleramide:mandelic acid co-crystal is characterized as having one, two, three, or four or more powder X-ray diffraction peaks including, but not limited to, 6.5, 9.9, 17.0, 17.6, 20.5, 21.0, 24.7, and 26.5 degrees 2-theta. selected from the group consisting of approximately 6.5, 9.9, 12.0, 13.2, 16.7, 17.0, 17.6, 18.2, 19.9, 20.5, 21.0, 22.7, 24.7, 25.5, 26.5, 29.8, 33.4, and 34.5 degrees 2-theta. In another embodiment, the isovaleramide:mandelic acid co-crystal is characterized as having five or more powder X-ray diffraction peaks including, but not limited to, 6.5, 9.9, 17.0, 17.6, 20.5, 21.0, 24.7, and 26.5 degrees 2-theta. In another embodiment, the isovaleramide:mandelic acid co-crystal is characterized as having a powder X-ray diffractogram comprising peaks at about 6.5, 9.9, 12.0, 13.2, 16.7, 17.0, 17.6, 18.2, 19.9, 20.5, 21.0, 22.7, 24.7, 25.5, 26.5, 29.8, 33.4, and 34.5 degrees 2-theta.

In a further embodiment, the isovaleramide:mandelic acid co-crystal has a powder X-ray diffraction pattern that is substantially the same as the pattern shown in FIG. 5G.

A further physical characteristic of the isovaleramide:mandelic acid co-crystal of the present invention includes its Raman spectrum. For example, certain embodiments of the isovaleramide:mandelic acid co-crystal of the present invention are characterized as having a Raman spectrum comprising peaks at about 3064, 2916, 1714, 1607, 1454, 1345, 1285, 1181, 1030, 1005, 969, 865, 840, 775, 737, 693, 619, 433, 366, and 146 cm⁻¹.

In a further embodiment, the isovaleramide:mandelic acid co-crystal has a Raman spectrum that is substantially the same as the pattern shown in FIG. 5E.

A further physical characteristic of the isovaleramide:mandelic acid co-crystal of the present invention includes its IR spectrum. For example, certain embodiments of the isovaleramide:mandelic acid co-crystal of the present invention are characterized as having an IR spectrum comprising peaks at about 2957, 1903, 1710, 1659, 1582, 1453, 1369, 1262, 1190, 1138, 1092, 1068, 932, 903, 728, and 698 cm⁻¹. In another embodiment, the isovaleramide:mandelic acid co-crystal is characterized as having a IR spectrum that is substantially the same as the pattern shown in FIG. 5C.

In yet a further embodiment, the isovaleramide:mandelic acid co-crystal is characterized as, having an endothermic transition at about 106° C. In yet a further embodiment the isovaleramide:mandelic acid co-crystal is characterized as having an endothermic transition at about 105° C. For example, an isovaleramide:mandelic acid co-crystal exhibits a DSC thermogram substantially as shown in FIG. 5A or 5F.

In an alternative aspect, the isovaleramide:mandelic acid co-crystal is characterized as exhibiting a weight loss, as determined by TGA, substantially similar to that shown in FIG. 5B.

The isovaleramide:mandelic acid co-crystal in certain embodiments is about 95.00%, 96.00%, 97.00%, 98.00%, 99.00%, 99.90%, or 99.99% free of impurities including solvate molecules.

Novel isovaleramide forms of the invention include, but are not limited to:

1) an isovaleramide:citric acid co-crystal characterized by a powder X-ray diffraction pattern expressed in terms of 2-theta angles, and wherein the X-ray powder diffraction pattern comprises approximately the 2-theta angle values listed and illustrated in FIG. 1A herein;

2) an isovaleramide:gentisic acid co-crystal characterized by a powder X-ray diffraction pattern expressed in terms of 2-theta angles, and wherein the X-ray powder diffraction pattern comprises approximately the 2-theta angle values listed and illustrated in FIG. 2D or 2F herein;

3) an isovaleramide:glutaric acid co-crystal characterized by a powder X-ray diffraction pattern expressed in terms of 2-theta angles, and wherein the X-ray powder diffraction pattern comprises approximately the 2-theta angle values listed and illustrated in FIG. 3C herein;

4) an isovaleramide:maleic acid co-crystal characterized by a powder X-ray diffraction pattern expressed in terms of 2-theta angles, and wherein the X-ray powder diffraction pattern comprises approximately the 2-theta angle values listed and illustrated in FIG. 4C herein; and

5) an isovaleramide:mandelic acid co-crystal characterized by a powder X-ray diffraction pattern expressed in terms of 2-theta angles, and wherein the X-ray powder diffraction pattern comprises approximately the 2-theta angle values listed and illustrated in FIG. 5D or 5G herein.

Pharmaceutical dosage forms of the novel forms of isovaleramide can be administered orally, parenterally, by inhalation spray, topically, rectally, nasally, buccally, vaginally or via an implanted reservoir. Oral and parenteral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Other dosage forms include an intradermal dosage form, an intramuscular dosage form, a subcutaneous dosage form, and an intravenous dosage form.

Forms of isovaleramide can be administered by controlled- or delayed-release means. Controlled-release pharmaceutical products generally have a common goal of improving drug therapy over that achieved by their non-controlled release counterparts. Ideally, the use of an optimally designed controlled-release preparation in medical treatment is characterized by a minimum of API substance being employed to cure or control the condition in a minimum amount of time. Advantages of controlled-release formulations generally include: 1) extended activity of the API; 2) reduced dosage frequency; 3) increased patient compliance; 4) usage of less total API; 5) reduction in local or systemic side effects; 6) minimization of API accumulation; 7) reduction in blood level fluctuations; 8) improvement in efficacy of treatment; 9) reduction of potentiation or loss of API activity; and 10) improvement in speed of control of diseases or conditions. (Kim, Chemg-ju, Controlled Release Dosage Form Design, 2 Technomic Publishing, Lancaster, Pa.: 2000).

Topical dosage forms of the invention include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity optionally greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, optionally in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th ed., Mack Publishing, Easton, Pa. (1990).

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are optionally sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Transdermal and mucosal dosage forms of the invention include, but are not limited to, ophthalmic solutions, patches, sprays, aerosols, creams, lotions, suppositories, ointments, gels, solutions, emulsions, suspensions, or other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). Dosage forms suitable for treating mucosal tissues within the oral cavity can be formulated as mouthwashes, as oral gels, or as buccal patches. Further, transdermal dosage forms include “reservoir type” or “matrix type” patches, which can be applied to the skin and worn for a specific period of time to permit the penetration of a desired amount of active ingredient.

Like the amounts and types of excipients, the amounts and specific type of active ingredient in a dosage form may differ depending on factors such as, but not limited to, the route by which it is to be administered to patients. In a particular embodiment, the isovaleramide form for use in such a composition is an isovaleramide:citric acid co-crystal, an isovaleramide:gentisic acid co-crystal, an isovaleramide:glutaric acid co-crystal, an isovaleramide:maleic acid co-crystal, or an isovaleramide:mandelic acid co-crystal.

In one embodiment of the invention, a pharmaceutical composition (or medicament) comprising an isovaleramide form of the present invention is administered orally as needed in an amount of from about 0.1 mg to about 1000 mg isovaleramide or from about 0.5 mg to about 500 mg isovaleramide. The dosage amounts can be administered in single or divided doses.

In other embodiments, the present invention is directed to compositions comprising an isovaleramide form as described herein and one or more diluents, carriers, and/or excipients suitable for the administration to a mammal for the treatment or prevention of one or more of the conditions described herein.

Typical pharmaceutical compositions and dosage forms comprise one or more excipients. Suitable excipients are well known to those skilled in the art of pharmacy, and non-limiting examples of suitable excipients are provided herein. For example, excipients that can be used in oral dosage forms of the invention include, but are not limited to, binders, stabilizers, fillers, disintegrants, and lubricants. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. In addition, pharmaceutical compositions or dosage forms may contain one or more compounds that reduce or alter the rate by which the active ingredient will decompose. Such compounds, which are referred to herein as “stabilizers”, include, but are not limited to, antioxidants, pH buffers, or salt buffers.

The novel isovaleramide forms of the invention are effective in the treatment or prevention of a number of illnesses and medical conditions, including, for example, epilepsy, migraine, Parkinson's disease, bipolar disorder, depression, schizophrenia, CNS disorders, and anxiety.

The precise dosage of the isovaleramide forms, as described herein, for the above methods of treatment or prevention may vary depending on the condition being treated/prevented, the symptoms of the condition, the patient being treated (e.g., age, weight, sex, etc.), and other factors.

In each of these methods of treatment or prevention, compositions to be administered include, but are not limited to, those comprising an isovaleramide:citric acid co-crystal, an isovaleramide:gentisic acid co-crystal, an isovaleramide:glutaric acid co-crystal, an isovaleramide:maleic acid co-crystal, an isovaleramide:mandelic acid co-crystal, or any combination thereof.

Isovaleramide can be made or prepared using various methods known to those skilled in the art. See, e.g., U.S. Pat. No. 5,763,494. Of course, other methods known to one of ordinary skill in the art may be used to prepare the active ingredient of isovaleramide.

Forms of the invention including, but not limited to, isovaleramide co-crystals, may be prepared by reacting the isovaleramide free base with an appropriate acid, such as an organic or inorganic acid, including without limitation, oxalic acid, succinic acid, malic acid, hydrochloric acid, sulfuric acid, fumaric acid, phosphoric acid, tartaric acid, maleic acid, malonic acid, adipic acid, and benzenesulfonic acid. For example, the process for forming a co-crystal can be carried out in a crystallization solvent in which both reactants (isovaleramide free base and acid) are sufficiently soluble.

In one embodiment, the present invention provides an isovaleramide co-crystal, comprising:

1) providing isovaleramide;

2) providing a co-crystal former; and

3) grinding, heating, or contacting in solution the isovaleramide with the co-crystal former under crystallization conditions.

In a still further embodiment, the present invention provides a method of making a pharmaceutical composition, comprising:

1) grinding, heating, or contacting in solution an isovaleramide with a co-crystal former, under crystallization conditions, so as to form a solid phase;

2) isolating an isovaleramide co-crystal comprising the isovaleramide and the co-crystal former; and

3) incorporating the isovaleramide co-crystal into a pharmaceutical composition.

In another embodiment, the isovaleramide co-crystal is isolated using one or more known procedures.

The grinding can be performed manually or mechanically. For example, the grinding can be performed in a Mini-Bead Beater™. The grinding can be performed for various amounts of time. Suitable times include but are not limited to 5, 10, 15, 20, 25, 30, 35, 40, 45, 50, 55, and 60 minutes.

Suitable co-crystal formers include organic acids such as those acids as described herein. In one aspect, a carboxylic acid or a dicarboxylic acid is used. Alternatively, a dihydroxybenzoic acid is used. Suitable carboxylic acids include, but are not limited to, citric acid, maleic acid, mandelic acid, gentisic acid, and glutaric acid. Accordingly, in particular embodiments, the method is used to prepare an isovaleramide:citric acid co-crystal, an isovaleramide:gentisic acid co-crystal, an isovaleramide:glutaric acid co-crystal, an isovaleramide:maleic acid co-crystal, or an isovaleramide:mandelic acid co-crystal.

The ratio of isovaleramide to co-crystal former may vary. In certain embodiments, for example, the ratio of isovaleramide to co-crystal former is from about 1:10 to about 10:1. In other embodiments, the ratio of isovaleramide to co-crystal former is from about 1:5 to about 5:1, about 1:4 to about 4:1, about 1:3 to about 3:1, about 1:2 to about 2:1, or about 1:1.

In another embodiment, the present invention is directed to a method of making an isovaleramide carboxylic acid co-crystal, comprising:

1) combining isovaleramide, a carboxylic acid, and a solvent, to form a solution;

2) heating said solution; and

3) cooling said solution or allowing said solution to cool, wherein the isovaleramide carboxylic acid co-crystal is thereby formed.

The isovaleramide carboxylic acid co-crystal can be isolated using known methods. For example, the isovaleramide carboxylic acid co-crystal can be isolated using vacuum filtration or centrifuge filtration. The isolated isovaleramide carboxylic acid co-crystal can also be dried, for example under a vacuum, for various amounts of time, for example from 30 minutes to about 6 hours. In certain embodiments, the co-crystal is dried for 1 hour.

Suitable solvents may comprise methanol, ethanol, propanol, isopropanol, and the like.

The ratio of carboxylic acid to isovaleramide may vary. In certain embodiments, for example, the ratio of isovaleramide to carboxylic acid is from about 1:10 to about 10:1. In other embodiments, the ratio of isovaleramide to carboxylic acid is from about 1:5 to about 5:1, about 1:4 to about 4:1, about 1:3 to about 3:1, about 1:2 to about 2:1, or about 1:1. In a further embodiment, the ratio of isovaleramide to carboxylic acid is about 1.3:1.

The concentration of the isovaleramide in the solution prepared in steps (1) and/or (2) can vary. For example, the concentration of isovaleramide can be, in certain embodiments, from about 0.01 M (moles per liter) to about 100 M, or about 0.1 M to about 10 M. In certain embodiments, the concentration of the isovaleramide in the solution prepared in steps (1) and/or (2) is about 1, 2, 3, 4, 5, 6, 7, or 8 M.

The solution formed in step (1) is heated in step (2). The solution can be heated to a temperature of about 35° C. to about 100° C., depending on the particular solvent and carboxylic acid used. In certain embodiments, the solution is heated to about 40, 50, 60, or 70° C. In other embodiments, the precise temperature to which the solution is heated is not critical as long as substantially all of the isovaleramide and/or carboxylic acid is dissolved. Optionally, the solution may be stirred or mixed while it is being heated. In certain instances, the heated solution can be allowed to cool to room temperature without any particular cooling. In other instances, the heated solution may be actively cooled.

It is further understood that the solution formed in step (1) does not require that all of the isovaleramide and carboxylic acid be dissolved prior to heating.

Assaying for the presence of novel isovaleramide forms, including co-crystals of the isovaleramide and the co-crystal former and novel hydrate, solvates, co-crystals, and polymorphs thereof may be carried out by conventional methods known in the art. For example, it is convenient and routine to use powder X-ray diffraction techniques to assess the presence of the co-crystals. This may be affected by comparing the spectra of the isovaleramide, the co-crystal former, and the putative co-crystals in order to establish whether or not true co-crystals have been formed. Other techniques, used in an analogous fashion, include differential scanning calorimetry (DSC), thermogravimetric analysis (TGA) and Raman spectroscopy. Single crystal X-ray diffraction is especially useful in identifying co-crystal structures.

In a further aspect, the present invention therefore provides a method of screening for isovaleramide co-crystals, comprising:

1) providing (i) isovaleramide, and (ii) a co-crystal former;

2) screening for an isovaleramide co-crystal by subjecting each combination of isovaleramide and co-crystal former to a process comprising: (a) grinding, heating or contacting in solution the isovaleramide with the co-crystal former under crystallization conditions so as to form a solid phase; and (b) isolating co-crystals comprising the isovaleramide and the co-crystal former.

In additional embodiments, the isovaleramide forms of the invention include, but are not limited to:

1) an isovaleramide:citric acid co-crystal prepared by a grinding process as described herein;

2) an isovaleramide:gentisic acid co-crystal prepared by a grinding process as described herein;

3) an isovaleramide:gentisic acid co-crystal prepared by a recrystallization process as described herein;

4) an isovaleramide:glutaric acid co-crystal prepared by a grinding process as described herein;

5) an isovaleramide:maleic acid co-crystal prepared by a grinding process as described herein;

6) an isovaleramide:mandelic acid co-crystal prepared by a grinding process as described herein; and

7) an isovaleramide:mandelic acid co-crystal prepared by a recrystallization process as described herein.

The invention is described further in the following examples, which are illustrative and in no way limiting.

### EXEMPLIFICATION

### Materials and Methods

Some or all of the following materials and methods were used in the various experiments described in the examples disclosed herein.

### Analytical Equipment And Procedures

Thermogravimetric Analysis

Thermogravimetric analysis of each sample was performed using a Q500 Thermogravimetric Analyzer (TA Instruments, New Castle, Del., U.S.A.), which uses as its control software Advantage for QW-Series, version 1.0.0.78, Thermal Advantage Release 2.0 (©2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©1991-2001 TA Instruments-Water LLC).

For all of the experiments, the basic procedure for performing thermogravimetric analysis comprised transferring an aliquot of a sample into a platinum sample pan (Pan part # 952019.906; (TA Instruments, New Castle, Del. USA)). The pan was placed on the loading platform and was then automatically loaded into the Q500 Thermogravimetric Analyzer using the control software. Thermograms were obtained by individually heating the sample at 10° C./minute across a temperature range (generally from 25° C. to 300° C.) under flowing dry nitrogen (compressed nitrogen, grade 4.8 (BOC Gases, Murray Hill, N.J. USA)), with a sample purge flow rate of 60 mL/minute and a balance purge flow rate of 40 mL/minute. Thermal transitions (e.g., weight changes) were viewed and analyzed using the analysis software provided with the instrument.

Differential Scanning Calorimetry

DSC analysis of each sample was performed using a Q1000 Differential Scanning Calorimeter (TA Instruments, New Castle, Del., U.S.A.), which uses Advantage for QW-Series, version 1.0.0.78, Thermal Advantage Release 2.0 (©2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©2001 TA Instruments-Water LLC).

For all of the DSC analyses, an aliquot of a sample was weighed into an aluminum sample pan (Pan part # 900786.091; lid part # 900779.901 (TA Instruments, New Castle Del. USA)). The sample pan was sealed either by crimping for dry samples or press fitting for wet samples (such as hydrated or solvated samples). The sample pan was loaded into the Q1000 Differential Sanning Calorimeter, which is equipped with an autosampler, and a thermogram was obtained by individually heating the same using the control software at a rate of 10° C./minute from Tmin (typically 30° C.) to Tmax (typically 300° C.) using an empty aluminum pan as a reference. Dry nitrogen (compressed nitrogen, grade 4.8 (BOC Gases, Murray Hill, N.J. USA)) was used as a sample purge gas and was set at a flow rate of 50 mL/minute. Thermal transitions were viewed and analyzed using the analysis software provided with the instrument.

Powder X-Ray Diffraction

A powder X-ray diffraction (PXRD) pattern for the samples was obtained using a D/Max Rapid, Contact (Rigaku/MSC, The Woodlands, Tex., U.S.A.), which uses as its control software RINT Rapid Control Software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the PXRD analysis, the acquisition parameters were as follows: source was Cu with a K line at 1.5406 Å; x-y stage was manual; collimator size was 0.3 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-60 degrees; the integration chi range was 0-360 degrees; the number of chi segments was 1; the step size used was 0.02; the integration utility was cylint; normalization was used; dark counts were 8; omega offset was 180; and chi and phi offsets were 0.

PXRD diffractograms were also acquired via the Bruker AXS D8 Discover X-ray Diffractometer. This instrument was equipped with GADDS™ (General Area Diffraction Detection System), a Bruker AXS HI-STAR Area Detector at a distance of 15.05 cm as per system calibration, a copper source (Cu/K_{α} 1.54056 angstroms), automated x-y-z stage, and 0.5 mm collimator. The sample was compacted into pellet form and mounted on the x-y-z stage. A diffractogram was acquired under ambient conditions (25 degrees C.) at a powder setting of 40 kV and 40 mA in reflection mode while the sample remained stationary. The exposure time was varied and specified for each sample. The diffractogram obtained underwent a spatial remapping procedure to account for the geometrical pincushion distortion of the area detector then integrated along chi from −118.8 to −61.8 degrees and 2-theta 2.1-37 degrees at a step size of 0.02 degrees with normalization set to bin normalize.

The relative intensity of peaks in a diffractogram is not necessarily a limitation of the PXRD pattern because peak intensity can vary from sample to sample, e.g., due to crystalline impurities. Further, the angles of each peak can vary by about +/0.1 degrees, or +/−0.05. The entire pattern or most of the pattern peaks may also shift by about +/−0.1 degrees to about +/−0.2 degrees due to differences in calibration, settings, and other variations from instrument to instrument and from operator to operator. All reported PXRD peaks in the Figures, Examples, and elsewhere herein are reported with an error of about +0.1 degrees 2-theta.

For PXRD data herein, including Tables and Figures, each composition of the present invention may be characterized by any one, any two, any three, any four, any five, any six, any seven, or any eight or more of the 2-theta angle peaks. Any one, two, three, four, five, or six DSC transitions can also be used to characterize the compositions of the present invention. The different combinations of the PXRD peaks and the DSC transitions can also be used to characterize the compositions.

Raman Spectroscopy

An aliquot of the sample was transferred to a glass slide. The glass slide was positioned in the sample chamber. The measurement was made using an Almega™ Dispersive Raman (Almega™ Dispersive Raman, Thermo-Nicolet, 5225 Verona Road, Madison, Wis. 53711-4495) system fitted with a 785 nm laser source. The sample was manually brought into focus using the microscope portion of the apparatus with a 10× power objective (unless otherwise noted), thus directing the laser onto the surface of the sample. The spectrum was acquired using the parameters outlined in Table A. (Exposure times and number of exposures may vary; changes to parameters will be indicated for each acquisition.)

TABLE A
Raman Spectral acquisition parameters

| Parameter | Setting Used |
|---|---|
| Exposure time (s) | 2.0 |
| Number of exposures | 10 |
| Laser source wavelength (nm) | 785 |
| Laser power (%) | 100 |
| Aperture shape | pin hole |
| Aperture size (micrometer) | 100 |
| Spectral range | 104-3428 |
| Grating position | Single |
| Temperature at acquisition | 24.0 |
| (degrees C.) | |

IR acquisitions

IR spectra were obtained using Nexus™ 470 FT-IR, Thermo-Nicolet, 5225 Verona Road, Madison, Wis. 53711-4495 and were analyzed with Control and Analysis software: OMNIC, Version 6.0a, (C) Thermo-Nicolet, 1995-2004.

### Single Crystal X-Ray Analysis

Single crystal x-ray crystallographic analyses conducted in connection with the experiments described herein were used to determine unit cell dimensions, space group, and atomic position of all atoms in a compound relative to the origin of its unit cell. The unit cell dimension is defined by three parameters; length of the sides of the cell, relative angles of sides to each other and the volume of the cell. The lengths of the sides of the unit cell are defined by a, b and c. The relative angles of the cell sides are defined by alpha, beta, and gamma. The volume of the cell is defined as V. A more detailed account of unit cells can be found in Chapter 3 of Stout & Jensen, X-Ray Structure Determination; A Practical Guide, Mac Millian Co., New York, N.Y. (1968).

Single crystal x-ray data were collected on a Bruker SMART-APEX CCD diffractometer (M. J. Zaworotko, Department of Chemistry, University of South Florida). Lattice parameters were determined from least squares analysis. Reflection data was integrated using the program SAINT. The structure was solved by direct methods and refined by fill matrix least squares using the program SHELXTL (Sheldrick, G. M. SHELXTL, Release 5.03; Siemans Analytical X-ray Instruments Inc.: Madison, Wis.).

The isovaleramide forms of the present invention can be characterized, e.g., by the TGA or DSC data or by any one, any two, any three, any four, any five, any six, any seven, any eight, any nine, any ten, or any single integer number of PXRD 2-theta angle peaks as described above, Raman shift peaks, Infrared spectroscopy peaks (e.g., IR, FTIR), or by single crystal x-ray diffraction data.

### Example 1

### Isovaleramide:Citric Acid Co-Crystal

Isovaleramide (10.9 mg, 0.108 mmol) and citric acid anhydrous (20.3 mg, 0.106 mmol) were ground in a Mini-Bead Beater for 10 minutes. The resulting product was characterized using PXRD, IR, and Raman, as shown in FIGS. 1A, 1B, and 1C, respectively.

### Example 2

### Isovaleramide:Gentisic Acid Co-Crystal

Isovaleramide (49.3 mg, 0.487 mmol), gentisic acid (58.9 mg, 0.382 mmol), and methanol (0.120 mL) were combined and heated to 60 degrees C. to form a solution. Once all solids were dissolved the solution was left to cool to room temperature. Upon cooling, space filling needle-shaped crystals formed that were collected in a centrifuge filter and dried under a vacuum for 1 hour. The resulting product was characterized using DSC, TGA, IR, PXRD, and Raman, as shown in FIGS. 2A, 2B, 2C, 2D and 2E, respectively.

### Example 3

### Isovaleramide:Gentisic Acid Co-Crystal

Isovaleramide (10.6 mg, 0.1050 mmol) and gentisic acid (15.7 mg, 0.102 mmol) were ground in a Mini-Bead Beater for 10 minutes. The resulting product was characterized using PXRD, DSC, IR, and Raman, as shown in FIGS. 2F, 2G, 2H, and 2I, respectively.

### Example 4

### Isovaleramide:Glutaric Acid Co-Crystal

Isovaleramide (10.5 mg, 0.104 mmol) and glutaric acid (14.0 mg, 0.106 mmol) were ground in a Mini-Bead Beater for 16 minutes. The resulting product was then characterized using DSC, IR, PXRD, and Raman, the results of which are shown in FIGS. 3A, 3B, 3C, and 3D, respectively.

### Example 5

### Isovaleramide:Maleic Acid Co-Crystal

Isovaleramide (9.2 mg, 0.091 mmol) and maleic acid (13.4 mg, 0.115 mmol) were ground in a Mini-Bead Beater for 10 minutes. The resulting product was then characterized using DSC, IR, PXRD, and Raman, and the results of which are shown in FIGS. 4A, 4B, 4C, and 4D, respectively.

### Example 6

### Isovaleramide:Mandelic Acid Co-Crystal

Isovaleramide (56.0 mg, 0.554 mmol), mandelic acid (130.4 mg, 0.857 mmol) and methanol (0.08 mL) were combined and heated to 60 degrees C. to form a solution. Once all solids were dissolved the solution was left to cool to room temperature. Upon cooling, needle shaped crystals formed that were collected in a centrifuge filter and dried under a vacuum for 1 hour. The resulting product was then characterized using DSC, TGA, IR, PXRD, and Raman, the results of which are shown in FIGS. 5A, 5B, 5C, 5D, and 5E, respectively.

### Example 7

### Isovaleramide:Mandelic Acid Co-Crystal

Isovaleramide (9.55 mg, 0.094 mmol) and mandelic acid (16.0 mg, 0.105 mmol) were ground in a Mini-Bead Beater for 10 minutes. The resulting product was then characterized using DSC and PXRD, the results of which are shown in FIGS. 5F and 5G, respectively.

### Example 8

### Isovaleramide:Gentisic Acid Co-crystal Single-Crystal X-ray Data

Single-crystal x-ray data: C₁₂H₁₇NO₅, M=255.27, monoclinic P2(1)/c; a=11.806(2) Å, b=9.9045(17) Å, c=11.387(2) Å, alpha=90°, beta=102.848(3)°, gamma=90°, T=100(2) K, Z=4, D_{c}=1.306 g/cm³, V=1298.2(4) Å³, wavelength=0.71073 Å.

Having now fully described this invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entireties.

1-46. (canceled)

47. A co-crystal comprising isovaleramide and a co-crystal former, wherein the co-crystal former is selected from the group consisting of citric acid, gentisic acid, glutaric acid, maleic acid, and mandelic acid.

48. The co-crystal of claim 47, wherein said co-crystal former is citric acid and wherein said co-crystal exhibits a powder X-ray diffraction pattern comprising a peak at about 5.0 degrees 2-theta.

49. The co-crystal of claim 47, wherein said co-crystal former is citric acid and wherein said co-crystal exhibits a powder X-ray diffraction pattern comprising peaks at about 5.0 and about 20.2 degrees 2-theta.

50. The co-crystal of claim 47, wherein said co-crystal former is citric acid and wherein said co-crystal exhibits a powder X-ray diffraction pattern comprising peaks at about 5.0, 15.3, 15.7, and about 20.2 degrees 2-theta.

51. The co-crystal of claim 47, wherein said co-crystal former is gentisic acid and wherein said co-crystal exhibits a powder X-ray diffraction pattern comprising peaks at about 7.4, 15.0, and about 15.9 degrees 2-theta.

52. The co-crystal of claim 47, wherein said co-crystal former is gentisic acid and wherein said co-crystal exhibits a powder X-ray diffraction pattern comprising peaks at about 19.1, 23.0, and about 25.7 degrees 2-theta.

53. The co-crystal of claim 47, wherein said co-crystal former is gentisic acid and wherein said co-crystal exhibits a powder X-ray diffraction pattern comprising peaks at about 7.4, 15.0, 15.9, 19.1, 23.0, and about 25.7 degrees 2-theta.

54. The co-crystal of claim 47, wherein said co-crystal former is glutaric acid and wherein said co-crystal exhibits a powder X-ray diffraction pattern comprising peaks at about 9.1, 22.8, and about 23.5 degrees 2-theta.

55. The co-crystal of claim 47, wherein said co-crystal former is glutaric acid and wherein said co-crystal exhibits a powder X-ray diffraction pattern comprising peaks at about 8.3, 19.7, and about 21.6 degrees 2-theta.

56. The co-crystal of claim 47, wherein said co-crystal former is glutaric acid and wherein said co-crystal exhibits a powder X-ray diffraction pattern comprising peaks at about 8.3, 9.1, 19.7, 21.6, 22.8, and about 23.5 degrees 2-theta.

57. The co-crystal of claim 47, wherein said co-crystal former is maleic acid and wherein said co-crystal exhibits a powder X-ray diffraction pattern comprising peaks at about 8.6, 18.5, and about 21.5 degrees 2-theta.

58. The co-crystal of claim 47, wherein said co-crystal former is maleic acid and wherein said co-crystal exhibits a powder X-ray diffraction pattern comprising peaks at about 20.2, 23.0, and about 26.2 degrees 2-theta.

59. The co-crystal of claim 47, wherein said co-crystal former is maleic acid and wherein said co-crystal exhibits a powder X-ray diffraction pattern comprising peaks at about 8.6, 18.5, 20.2, 21.5, 23.0 and about 26.2 degrees 2-theta.

60. The co-crystal of claim 47, wherein said co-crystal former is mandelic acid and wherein said co-crystal exhibits a powder X-ray diffraction pattern comprising peaks at about 6.5, 9.9, and about 17.6 degrees 2-theta.

61. The co-crystal of claim 47, wherein said co-crystal former is mandelic acid and wherein said co-crystal exhibits a powder X-ray diffraction pattern comprising peaks at about 20.5, 21.0, and about 24.7 degrees 2-theta.

62. The co-crystal of claim 47, wherein said co-crystal former is mandelic acid and wherein said co-crystal exhibits a powder X-ray diffraction pattern comprising peaks at about 6.5, 9.9, 17.6, 20.5, 21.0, and about 24.7 degrees 2-theta.

63. A method of treating or preventing epilepsy, migraine, Parkinson's disease, bipolar disorder, depression, schizophrenia, or anxiety, comprising administering an effective amount of a co-crystal, wherein the co-crystal comprises isovaleramide and a co-crystal former, and further wherein the co-crystal former is selected from the group consisting of citric acid, gentisic acid, glutaric acid, maleic acid, and mandelic acid.

64. A pharmaceutical composition comprising a co-crystal, wherein the co-crystal comprises isovaleramide and a co-crystal former, and further wherein the co-crystal former is selected from the group consisting of citric acid, gentisic acid, glutaric acid, maleic acid, and mandelic acid.
